(19) United States
(12) Patent Application Publication
(10) Pub. No.: US 2012/0265007 A1
(43) Pub. Date: Oct. 18, 2012

(54) ENDOSCOPE

(75) Inventors: Hiroki MORIYAMA, Akishima-shi (JP); Ken SHIGETA, Hachioji-shi (JP); Kazuo BANJU, Hachioji-shi (JP)

(73) Assignee: OLYMPUS MEDICAL SYSTEMS CORP., Tokyo (JP)

(21) Appl. No.: 13/415,276

(22) Filed: Mar. 8, 2012

Related U.S. Application Data
(63) Continuation of application No. PCT/JP2011/058827, filed on Apr. 7, 2011.
(60) Provisional application No. 61/368,305, filed on Jul. 28, 2010.

(51) Int. Cl. A61B 1/01; A61B 1/018; A61B 1/05

(52) U.S. Cl. 600/104

(57) ABSTRACT

An endoscope includes a driving portion which drives a bending portion and a degree of displacement calculating portion which calculates a degree of displacement of a distal end of the insertion portion when the distal end of the insertion portion is displaced by reaction force generated by a movement of a distal end of a surgical instrument projecting from the distal end of the insertion portion. The endoscope further includes a control portion which controls a driving amount of the driving portion to eliminate the displacement in accordance with the degree of displacement calculated by the degree of displacement calculating portion.

### CROSS-REFERENCE TO RELATED APPLICATIONS

This application is a Continuation Application of PCT Application No. PCT/JP2011/058827, filed Apr. 7, 2011 and based upon and claiming the benefit of U.S. Provisional Application No. 61/368305, filed Jul. 28, 2010, the entire contents of all of which are incorporated herein by reference.

### BACKGROUND OF THE INVENTION

1. Field of the Invention

The present invention relates to an endoscope having at least three the bending portions.

2. Description of the Related Art

In general, an endoscope has a the bending portion to bring a distal end of an insertion portion closer to a target.

For example, Jpn. Pat. Appin. KOKAI Publication No. 5-211991 has disclosed an endoscope apparatus having one the bending portion. In the endoscope apparatus, one the bending portion bent so that the distal end of an insertion portion approaches a target.

### BRIEF SUMMARY OF THE INVENTION

According to an aspect of embodiments, an endoscope comprising: an insertion portion having a longitudinal axis; a bending portion which is provided at the distal end of the insertion portion and which is configured to bend; a holding portion which is provided closer to the distal end of the insertion portion than the bending portion and which holds the distal end of a surgical instrument so that the distal end of the surgical instrument is configured to project from the distal end of the insertion portion; a driving portion which drives the bending portion; a degree of displacement calculating portion which calculates a degree of displacement of the distal end of the insertion portion when the distal end of the insertion portion is displaced by reaction force generated by the movement of the distal end of the surgical instrument projecting from the distal end of the insertion portion; and a control portion which controls a driving amount of the driving portion to eliminate the displacement in accordance with the degree of displacement calculated by the degree of displacement calculating portion.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic configuration diagram of an endoscope system according to the present invention;

FIG. 2A is a perspective view of a node ring;

FIG. 2B is a diagram showing node rings arrayed in a the bending portion;

FIG. 3 is a diagram showing the configuration of a second bending portion;

FIG. 4 is a graph illustrating a maximum bending angle;

FIG. 5 is a diagram showing the configuration of a control unit;

FIG. 6A is a diagram illustrating how the bending portion is bent when a target is imaged;

FIG. 6B is a diagram illustrating how the bending portion is bent when the target is imaged;

FIG. 6C is a diagram illustrating how the bending portion is bent when the target is imaged;

FIG. 6D is a diagram illustrating how the bending portion is bent when the target is imaged;

FIG. 7A is a diagram illustrating how the bending portion is bent when a surgical instrument moves;

FIG. 7B is a diagram illustrating how the bending portion is bent when the surgical instrument moves;

FIG. 7C is a diagram illustrating how the bending portion is bent when the surgical instrument moves;

FIG. 7D is a diagram illustrating how the bending portion is bent when the surgical instrument moves;

FIG. 8A is a diagram illustrating how the bending portion is bent when the surgical instrument is inserted into a surgical instrument insertion channel;

FIG. 8B is a diagram illustrating how the bending portion is bent when the surgical instrument is inserted into the surgical instrument insertion channel;

FIG. 9A is a diagram illustrating how a control portion controls when the bending portion bent;

FIG. 9B is a diagram illustrating how the control portion controls when the bending portion bent;

FIG. 10A is a diagram illustrating an operating method of the whole endoscope;

FIG. 10B is a diagram illustrating the operating method of the whole endoscope;

FIG. 10C is a diagram illustrating the operating method of the whole endoscope;

FIG. 10D is a diagram illustrating the operating method of the whole endoscope;

FIG. 11 is a flowchart illustrating the operating method of the whole endoscope;

FIG. 12 is a flowchart illustrating how the bending portion is bent when the target is imaged;

FIG. 13 is a flowchart illustrating how the bending portion is bent;

FIG. 14 is a flowchart illustrating how the bending portion is bent when the surgical instrument moves;

FIG. 15 is a flowchart illustrating how the bending portion is bent when the surgical instrument is inserted into the surgical instrument insertion channel; and

FIG. 16 is a flowchart illustrating how the control portion controls when the bending portion bent.

### DETAILED DESCRIPTION OF THE INVENTION

Hereinafter, an embodiment of the present invention will be described in detail with reference to the drawings.

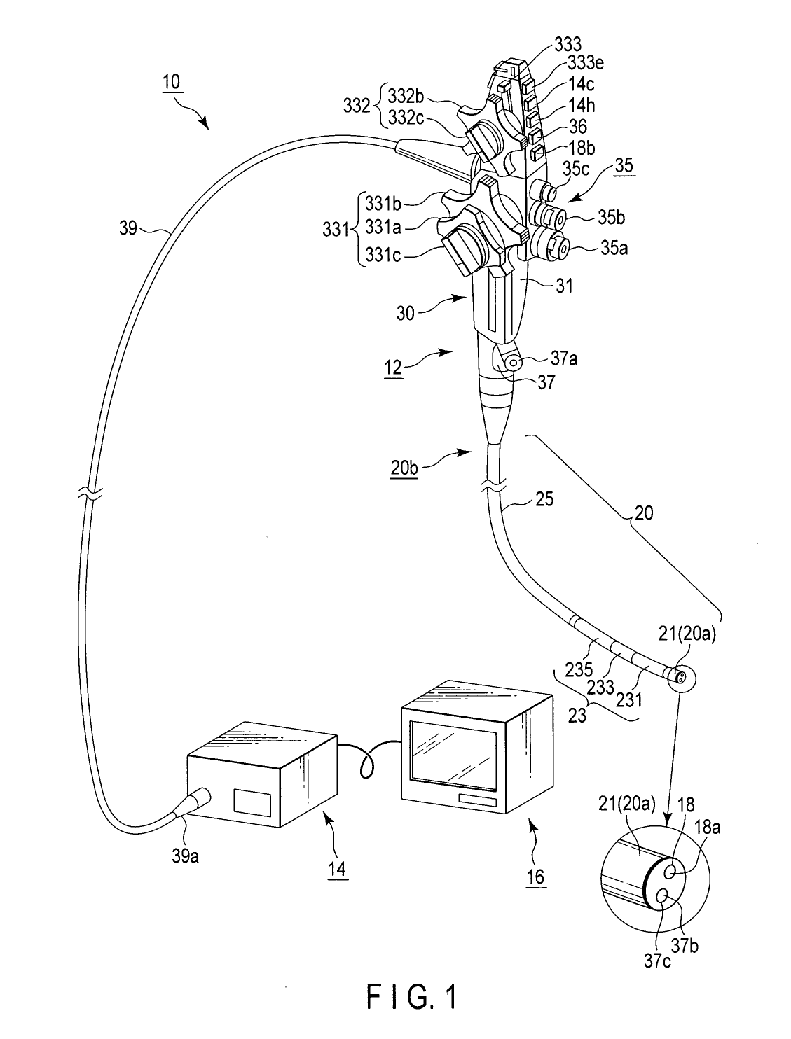

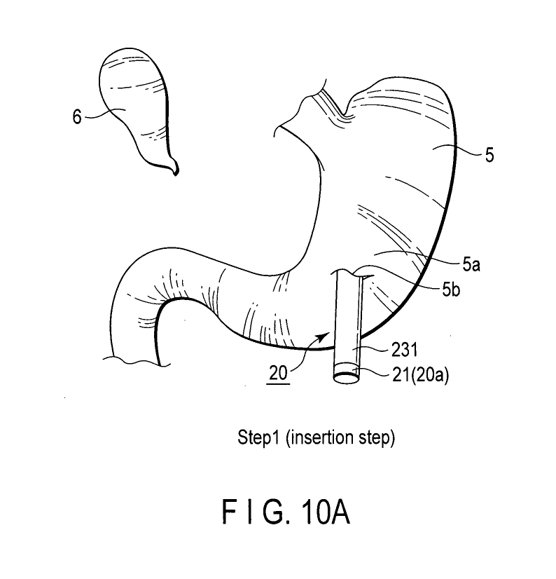

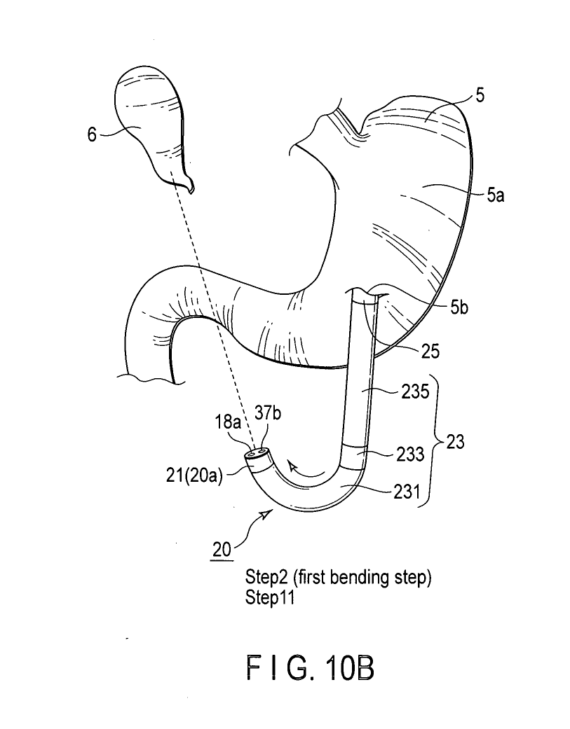

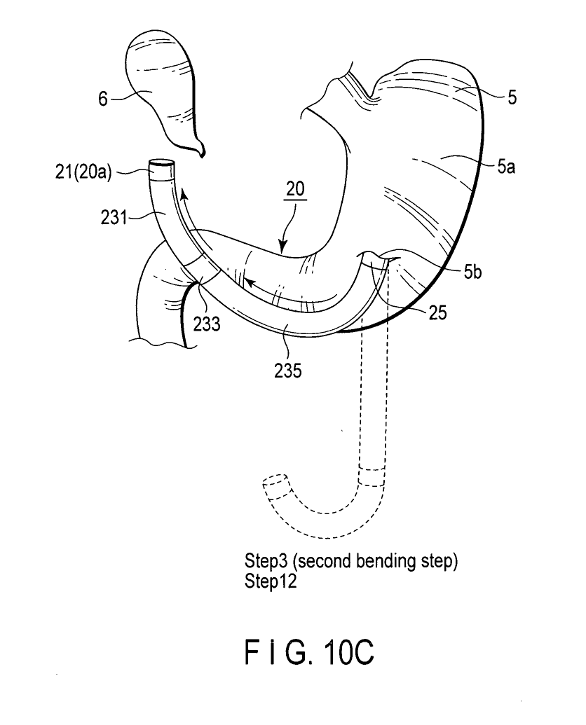

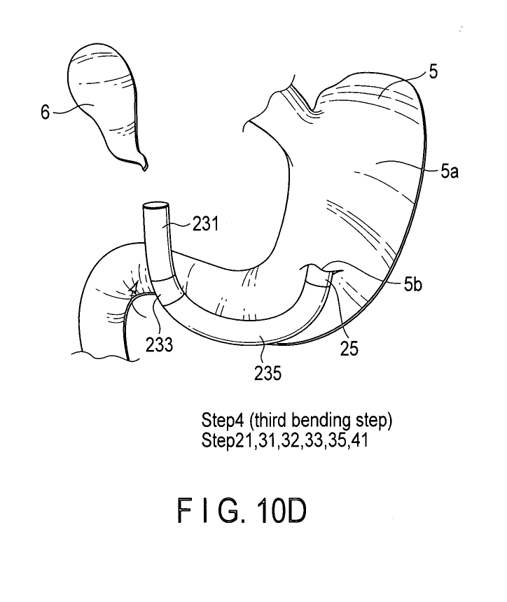

An endoscope system 10 shown in FIG. 1 comprises an endoscope 12 which images, for example, a target 6 shown in FIG. 10A, a control unit 14 (for example, a video processing portion) removably connected to the endoscope 12, and a monitor 16 which is connected to the control unit 14 and which is a display unit for displaying the target 6 imaged by the endoscope 12. The target 6 is, for example, gallbladder in a body cavity (abdominal cavity).

Such an endoscope system 10 is used, for example, in a surgical operation called natural orifice translumenal endscopic surgery (NOTES) for conducting cholecystectomy.

The endoscope 12 comprises a hollow elongate insertion portion 20 to be inserted into a body cavity, and an operation portion 30 which is provided at the proximal end of the insertion portion 20 and which operates the insertion portion 20.

The insertion portion 20 comprises a distal rigid portion 21, a bending portion 23, and a flexible tube 25, from the distal end of the insertion portion 20 to the proximal end of the insertion portion 20. The proximal end of the distal rigid portion 21 is coupled to the distal end of the bending portion 23, and the proximal end of the bending portion 23 is coupled to the distal end of the flexible tube 25.

The distal rigid portion 21 is a distal end 20a of the insertion portion 20, and is the distal end of the endoscope 12. The distal rigid portion 21 comprises an imaging surface 18a of an imaging unit 18 described later, and a distal opening portion portion 37c of a surgical instrument insertion channel 37b described later.

The bending portion 23 comprises a the bending portion 231 which is a first bending portion, a the bending portion 233 which is a second bending portion, and a the bending portion 235 which is a third bending portion, from the side of the distal rigid portion 21 to the side of the flexible tube 25. The endoscope 12 has only to comprise at least three the bending portions 231, 233, and 235 which bend in the insertion portion 20 to be inserted into a body cavity. Thus, the endoscope 12 is a multistage bending endoscope.

The bending portion 231 is provided closer to the distal end 20a of the insertion portion 20 than the bending portion 233 and the bending portion 235. The distal end of the bending portion 231 is coupled to the proximal end of the distal rigid portion 21. The distal end of the bending portion 233 is coupled to the proximal end of the bending portion 231. The distal end of the bending portion 235 is coupled to the proximal end of the bending portion 233. The proximal end of the bending portion 235 is coupled to the distal end of the flexible tube 25.

The bending portion 235 is longer than the bending portion 231 and the bending portion 233. The bending portion 231 is longer than the bending portion 233. The length of the bending portion 231 is, for example, 85 mm. The length of the bending portion 233 is, for example, 45 mm. The length of the bending portion 235 is, for example, 90 mm.

The bending portion 231 bent, for example, vertically and horizontally. The bending portion 233 bent, for example, vertically. The bending portion 235 bent, for example, horizontally.

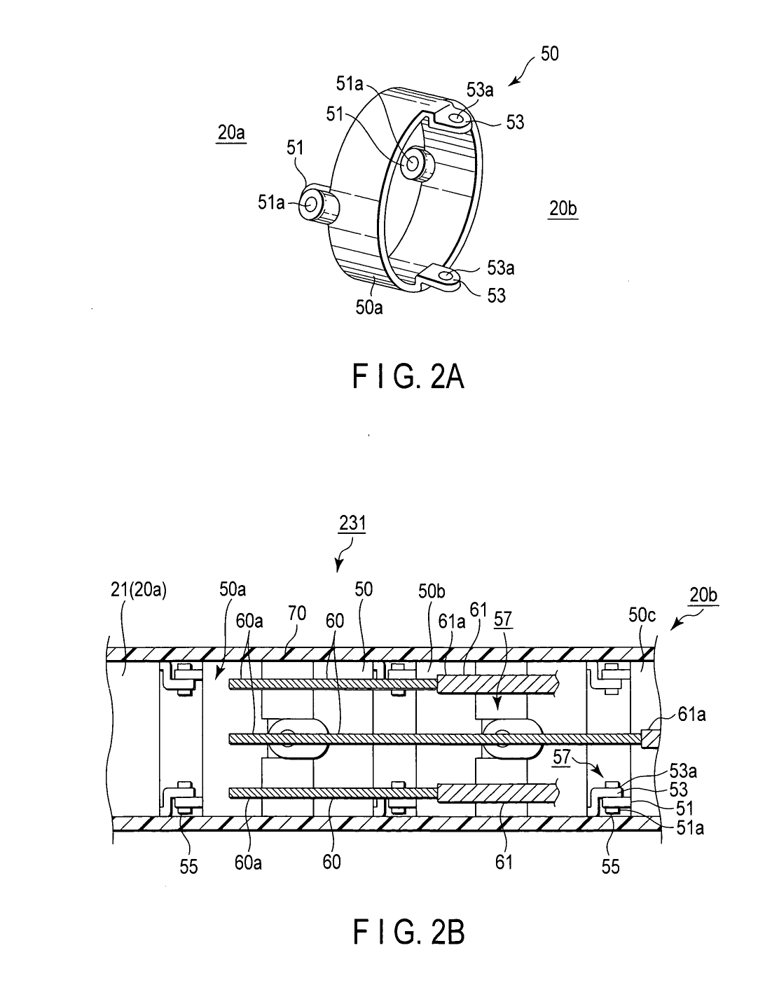

Now, the configurations of the bending portion 231, the bending portion 233, and the bending portion 235 are described with reference to FIG. 2A and FIG. 2B. The bending portion 231, the bending portion 233, and the bending portion 235 have substantially the same configuration. Therefore, the bending portion 231 is described by way of example.

The bending portion 231 has, for example, a hollow shape. The bending portion 231 comprises node rings 50 shown in FIG. 2A. The node rings 50 have a substantially cylindrical shape.

The node rings 50 are arrayed along the insertion (longitudinal axis) direction of the insertion portion 20 as shown in FIG. 2B. The node rings 50 adjacent to each other (located in turn along the insertion direction of the insertion portion 20) are pivotally coupled by a coupling portion 57, respectively. The coupling portion 57 will be described later. The node rings 50 are made of a rigid material such as a metal. The node rings 50 are formed, for example, by a metal thin plate pressed article or a forged article.

Two projecting pieces (front hinge mounts) 51 are disposed on the distal end (left side in FIG. 2A) of the node ring 50. The projecting piece 51 is formed by a flat part of the node ring 50 projecting forward (to the side of the distal end 20a of the insertion portion 20). The projecting piece 51 has a through-hole 51a bored therein. The two projecting pieces 51 are located substantially 180° apart from each other in the circumferential direction of the node ring 50.

Two projecting pieces (rear hinge mounts) 53 are also disposed on the rear end (right side in FIG. 2A) of the node ring 50. The projecting piece 53 is formed by a flat part of the node ring 50 projecting rearward (to the side of a proximal end 20b of the insertion portion 20). The projecting piece 53 is provided with a step substantially corresponding to the thickness of the projecting piece 51. The projecting piece 53 also has a through-hole 53a bored therein. The two projecting pieces 53 are located substantially 180° apart from each other in the circumferential direction of the node ring 50.

The front two projecting pieces 51 and the rear two projecting pieces 53 are located substantially 90° apart from each other in the circumferential direction of the node ring 50.

As shown in FIG. 2B, a rivet 55 which is a pivot member (pivot shaft) is inserted into the through-holes 51a and 53a in the projecting piece 53 of the node ring 50 on the side of the distal end 20a and in the projecting piece 51 of the node ring 50 on the side of the proximal end 20b. As a result, the node ring 50 on the side of the distal end 20a and the node ring 50 on the side of the proximal end 20b are coupled to each other via the rivet 55, and are pivotally supported to be able to turn around the rivet 55. In this way, a support shaft portion that uses the rivet 55 as a pivot support shaft is formed between the projecting piece 51 and the projecting piece 53. The rivet 55 thus couples the node rings 50 together.

In other words, the projecting piece 51, the projecting piece 53, and the rivet 55 are the coupling portion 57 which couple the node ring 50 on the side of the distal end 20a to the node ring 50 on the side of the proximal end 20b.

When the node ring 50 on the side of the distal end 20a is coupled to the node ring 50 on the side of the proximal end 20b via the rivets 55, the projecting piece 51 of the node ring 50 on the side of the proximal end 20b is stacked on the projecting piece 53 of the node ring 50 on the side of the distal end 20a.

In the bending portion 231 according to the present embodiment, the rivets 55 are alternately staggered substantially 90° from each other between the node rings 50. Thus, the bending portion 231 is configured to be able to bend, for example, in four vertical and horizontal directions.

The bending portion 233 bent vertically, so that the coupler 57 on the distal end of the bending portion 233 and the coupler 57 on the proximal end of the bending portion 233 are not alternately staggered 90° from each other in the circumferential direction of the node ring 50, and are aligned. The distal end of an operation wire 60 for bending the bending portion 233 is connected to the node ring 50 provided closest to the bending portion 231 in the bending portion 233.

The bending portion 235 bent horizontally, so that the coupler 57 on the distal end of the bending portion 235 and the coupler 57 on the proximal end of the bending portion 235 are not alternately staggered 90° from each other in the circumferential direction of the node ring 50, and are aligned. The distal end of the operation wire 60 for bending the bending portion 235 is connected to the node ring 50 provided closest to the bending portion 233 in the bending portion 235.

The coupler 57 of the bending portion 233 and the coupler 57 of the bending portion 235 are staggered 90° from each other in the circumferential direction of the node ring 50.

The node ring 50a provided closest to the distal rigid portion 21 is coupled to the distal rigid portion 21.

The bending portion 231 is connected to a later-described bending operation portion 331 of the operation portion 30 by the operation wire 60 inserted through the flexible tube 25. The bending portion 231 bent in desired directions, for example, vertically and horizontally when the operation wire 60 is pulled by the operation of the bending operation portion 331. As a result of the bend of the bending portion 231, the position and direction of the distal rigid portion 21 change, and the target 6 is captured in the imaging surface 18a and illuminated with illumination light.

As shown in FIG. 2B, a distal end 60a of the operation wire 60 is connected to the node ring 50a provided closest to the distal rigid portion 21 in the bending portion 231. The operation wire 60 is inserted through a guide sheath 61 (coil pipe). The guide sheath 61 is made of a flexible and elastic member. The guide sheath 61 is, for example, a tightly wound coil or a resin tube, and has a sheath shape. The inner hole of the guide sheath 61 is a guide member which guides the movement direction of the operation wire 60. A distal end 61a of the guide sheath 61 is connected not to the node ring 50a to which the operation wire 60 guided by this guide sheath 61 is connected, but to the node ring 50 located closer to the proximal end than the node ring 50a.

In the bending portion 231, for example, the distal end 61a of the guide sheath 61 which guides the vertical operation wire 60 is connected to the third node ring 50b from the side of the distal rigid portion 21.

In the bending portion 231, for example, the distal end 61a of the guide sheath 61 which guides the horizontal operation wire 60 is connected to the fifth node ring 50c from the side of the distal rigid portion 21.

A distal end 61a of each guide sheath 61 is fixed to an unshown wire guide provided in each node ring 50. The guide sheath 61 may be indirectly fixed to the wire guide by using an unshown connecting tool such as a connection cap. The proximal end of the guide sheath 61 may be connected to the proximal end of the bending portion 23 (the distal end of the flexible tube 25).

Thus, the node rings 50b and 50c to which the distal end 61a of the guide sheath 61 is connected are not the node rings 50a to which the operation wire 60 guided by this guide sheath 61 is connected. The node rings 50b and 50c are located closer to the proximal end of the insertion portion 20 than the node ring 50a. Therefore, the operation wire 60 is inserted through the guide sheath 61 and guided up to the node rings 50b and 50c. Thus, the guide sheath 61 enables the operation wire 60 to avoid interference without directly contacting internal objects such as other operation wires 60 or other guide sheaths 61.

The operation wires 60 and the guide sheaths 61 in the bending portion 233 and the bending portion 235 are similarly arranged. The operation wire 60 in the bending portion 231, the operation wire 60 in the bending portion 233, and the operation wire 60 in the bending portion 235 are separate from one another. The same applies to the guide sheaths 61.

As shown in FIG. 2B, the distal rigid portion 21 and the bending portion 23 are covered with an envelope tube 70. This envelope tube 70 is made of a resin material and an elastic material such as rubber. The envelope tube 70 is in substantially the same shape (for example, a hollow shape or a cylindrical shape) as the distal rigid portion 21 and the bending portion 23. The envelope tube 70 may otherwise be made of an injection-molded elastic material including a thermoplastic elastomer (styrene, olefin, or urethane) material. The thermoplastic elastomer is not exclusively injection-molded and may be molded in various ways, for example, may be cast, extruded, or blown.

As shown in FIG. 1, the flexible tube 25 is a tubular member having desired flexibility and extending from the operation portion 30, and is bendable by external force.

As shown in FIG. 1, the operation portion 30 comprises an operation portion main body 31 which is a grip portion for gripping the endoscope 12, and a universal cord 39.

As shown in FIG. 1, the operation portion main body 31 has the bending operation portion 331 for bending the bending portion 231. The bending operation portion 331 comprises a horizontal bending operation knob 331a for horizontally bending the bending portion 231, a vertical bending operation knob 331b for vertically bending the bending portion 231, and a fixing knob 331c for fixing the position of the bent the bending portion 231.

The horizontal bending operation knob 331a is connected to an unshown horizontal bending operation mechanism driven by the horizontal bending operation knob 331a. The vertical bending operation knob 331b is connected to an unshown vertical bending operation mechanism driven by the vertical bending operation knob 331b. The vertical bending operation mechanism and the horizontal bending operation mechanism are provided in the operation portion 30.

The horizontal bending operation mechanism is connected to the proximal end of the operation wire 60 for the bending portion 231 inserted through the flexible tube 25 and the bending portion 23. The distal end 60a of this operation wire 60 is connected to the bending portion 231 (the node ring 50a provided closest to the distal rigid portion 21) as shown in FIG. 2B. The horizontal bending operation mechanism is, for example, a pulley.

The vertical bending operation mechanism is connected to the proximal end of the operation wire 60 for the bending portion 231 inserted through the flexible tube 25 and the bending portion 23. The operation wire 60 connected to the vertical bending operation mechanism is different from the operation wire 60 connected to the horizontal bending operation mechanism. The distal end 60a of the operation wire 60 is connected to the bending portion 231 (the node ring 50a provided closest to the distal rigid portion 21) as shown in FIG. 2B. The vertical bending operation mechanism is, for example, a pulley.

The horizontal bending operation knob 331a pulls the operation wire 60 via the horizontal bending operation mechanism. Thus, the horizontal bending operation knob 331a horizontally bent the bending portion 231 via the horizontal bending operation mechanism and the operation wire 60. The vertical bending operation knob 331b pulls the operation wire 60 via the vertical bending operation mechanism. Thus, the vertical bending operation knob 331b vertically bent the bending portion 231 via the vertical bending operation mechanism and the operation wire 60.

In this way, the bending portion 231 is vertically and horizontally bent by manual operation via the horizontal bending operation knob 331a and the vertical bending operation knob 331b.

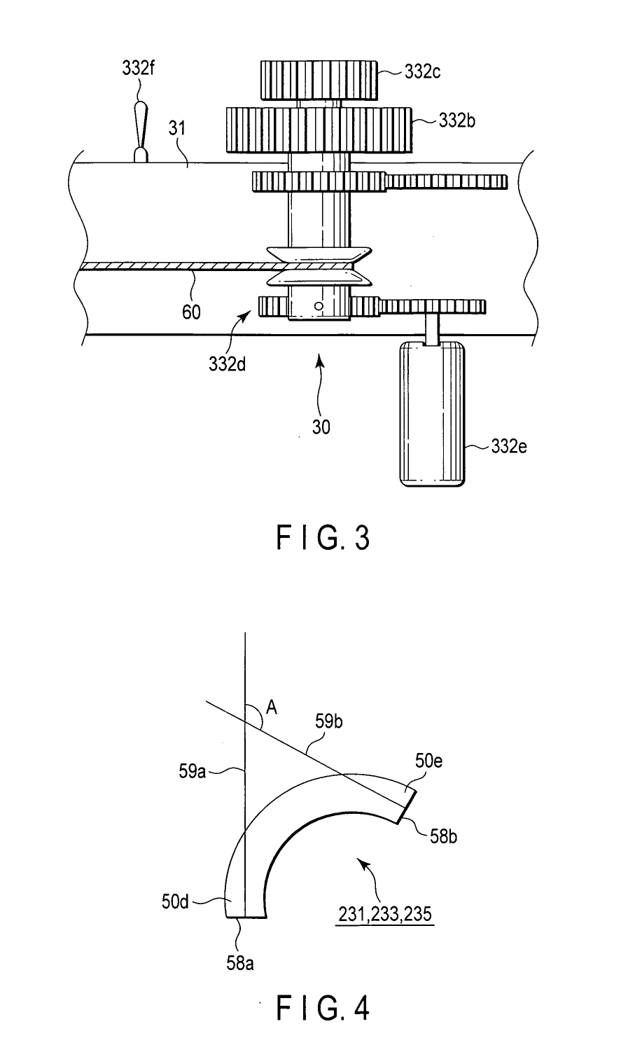

As shown in FIG. 1 and FIG. 3, the operation portion main body 31 has a bending operation portion 332 for bending the bending portion 233. The bending operation portion 332 comprises a vertical bending operation knob 332b for vertically bending the bending portion 233, and a fixing knob 332c for fixing the position of the bent the bending portion 233.

As shown in FIG. 3, the vertical bending operation knob 332b is connected to a vertical bending operation mechanism 332d driven by the vertical bending operation knob 332b. The vertical bending operation mechanism 332d is provided in the operation portion 30.

The vertical bending operation mechanism 332d is connected to the proximal end of the operation wire 60 for the bending portion 233 inserted through the flexible tube 25 and the bending portion 23. The distal end 60a of this operation wire 60 is connected to the bending portion 233, more specifically, to the node ring 50 provided closest to the bending portion 231 in the bending portion 233. The vertical bending operation mechanism 332d is, for example, a pulley.

The vertical bending operation knob 332b pulls the operation wire 60 via the vertical bending operation mechanism 332d. Thus, the vertical bending operation knob 332b vertically bent the bending portion 233 via the vertical bending operation mechanism 332d and the operation wire 60.

The bending portion 233 may be bent by electric operation.

In this case, as shown in FIG. 3, the operation portion main body 31 comprises a driving portion 332e which has driving force to electrically bend the bending portion 233 vertically and which is attachable to/detachable from the operation portion main body 31, and a driving operation portion 332f.

The driving portion 332e is, for example, a motor.

The driving operation portion 332f operates the driving portion 332e so that the driving force of the driving portion 332e is transmitted to the vertical bending operation mechanism 332d. The driving operation portion 332f also stops the transmission of the driving force of the driving portion 332e to the operation wire 60, and fixes the position of the bent the bending portion 233. The driving operation portion 332f is, for example, a switch.

The configuration of the vertical bending operation mechanism 332d shown in FIG. 3 is the same as the configurations of unshown bending operation mechanisms in the bending portions 231 and 235.

Thus, the bending portion 233 is electrically driven when the driving portion 332e is attached to the operation portion main body 31, and the bending portion 233 is switched to manual operation when the driving portion 332e is detached from the operation portion main body 31.

That is, the bending portion 233 is vertically bent by manual operation via the vertical bending operation knob 332b, or is vertically bent by electric operation via the driving portion 332e. In this way, the manual operation or the electric operation is selected in the bending portion 233, and the bending portion 233 is bent by the manual operation or the electric operation.

As shown in FIG. 1, the operation portion main body 31 also comprises a bending operation portion 333 for bending the bending portion 235, a driving portion 333e which has driving force to electrically bend the bending portion 235 horizontally and which is provided in the operation portion main body 31, and an unshown horizontal bending operation mechanism to which the driving force of the driving portion 333e is transmitted.

The bending operation portion 333 is provided in the vicinity of the horizontal bending operation knob 331a, the vertical bending operation knob 331b, and the vertical bending operation knob 332b. More specifically, the bending operation portion 333 is provided between the horizontal bending operation knob 331a, the vertical bending operation knob 331b, and the vertical bending operation knob 332b, and the exterior of the endoscope 12, in the thickness direction of the operation portion 30. When the horizontal bending operation knob 331a, the vertical bending operation knob 331b, and the vertical bending operation knob 332b are operated by one hand, the bending operation portion 333 is located to be operated by the thumb of this hand.

The bending operation portion 333 operates the driving portion 333e so that the driving force of the driving portion 333e is transmitted to the horizontal bending operation mechanism. The bending operation portion 333 also stops the transmission of the driving force of the driving portion 333e to the operation wire 60, and fixes the position of the bent the bending portion 235. The bending operation portion 333 is, for example, a switch.

The driving portion 333e is, for example, a motor.

The horizontal bending operation mechanism comprises, for example, a shaft and a pulley. The horizontal bending operation mechanism is driven by the driving force of the driving portion 333e. The horizontal bending operation mechanism is provided in the operation portion main body 31.

The horizontal bending operation mechanism is connected to the proximal end of the operation wire 60 for the bending portion 235 inserted through the flexible tube 25 and the bending portion 23. The distal end 60a of this operation wire 60 connected to the horizontal bending operation mechanism is connected to the bending portion 235 (the node ring 50 provided closest to the bending portion 233 in the bending portion 233).

When the bending operation portion 333 is operated, the driving portion 333e pulls the operation wire 60 by the driving force via the horizontal bending operation mechanism. As a result, the bending portion 235 horizontally bent via the driving portion 333e, the horizontal bending operation mechanism, and the operation wire 60.

In this way, the bending portion 235 is horizontally bent by the electric operation via the driving portion 333e.

Here, as shown in FIG. 4, the bending portions 231, 233, and 235 each comprises a proximal straight line 59a in the axial direction of its proximal end, and a distal straight line 59b in the axial direction of its distal end. Moreover, the bending portions 231, 233, and 235 each comprises a maximum bending angle A which represents an angle made between the proximal straight line 59a and the distal straight line 59b when each the bending portion bent. The proximal straight line 59a is a straight line perpendicular to the proximal end, and the distal straight line 59b is a straight line perpendicular to the plane of the distal end.

The plane in the diametrical direction of the node ring 50d is a proximal plane 58a. In this case, the proximal straight line 59a is perpendicular to the proximal plane 58a.

The plane in the diametrical direction of the node ring 50e is a proximal plane 58b. In this case, the distal straight line 59b is perpendicular to the proximal plane 58b.

When each of the bending portions 231, 233, and 235 is bent as shown in FIG. 4, the proximal straight line 59a intersects with the distal straight line 59b. The angle at the intersection of the proximal straight line 59a and the distal straight line 59b is the maximum bending angle A of the bending portions 231, 233, and 235.

The maximum bending angle of the bending portion 231 is, for example, 210°.

The maximum bending angle of the bending portion 233 is, for example, 80°.

The maximum bending angle of the bending portion 235 is, for example, 210°.

The maximum bending angle of the bending portion 231 is preferably, for example, 180°.

The maximum bending angle of the bending portion 233 is preferably, for example, 70°.

The maximum bending angle of the bending portion 235 is preferably, for example, 180°.

The maximum bending angle of the bending portion 231 is particularly preferably, for example, 150°.

The maximum bending angle of the bending portion 233 is particularly preferably, for example, 60°.

The maximum bending angle of the bending portion 235 is preferably, for example, 150°.

As described above, the maximum bending angle of the bending portion 231 is 150° or more and 210° or less when the bending portion 231 is bent. The maximum bending angle of the bending portion 233 is 60° or more and 80° or less when the bending portion 233 is bent. The maximum bending angle of the bending portion 235 is 150° or more and 210° or less when the bending portion 235 is bent.

As shown in FIG. 1, the operation portion main body 31 has a switch portion 35. The switch portion 35 is operated by the hand of a surgeon when the surgeon grips the operation portion main body 31. The switch portion 35 comprises a suction switch 35a, an air/water supply switch 35b, and various buttons 35c for endoscope imaging. The suction switch 35a is operated when the endoscope 12 sucks, for example, mucus from the distal rigid portion 21. The air/water supply switch 35b is operated when the endoscope 12 supplies air/water to secure a clean observation view field mainly in the distal rigid portion 21. The buttons 35c are operated when the target 6 is imaged via the imaging surface 18a in the imaging unit 18.

As shown in FIG. 1, the operation portion main body 31 comprises an operation portion 14c for operating a bending angle calculating portion 14a and a control portion 14b in the control unit 14 described later, and an operation portion 14h for setting and canceling a surgical instrument insertion/removal mode in the endoscope 12. Details of the operation portions 14c and 14h and the surgical instrument insertion/removal mode will be described later.

As shown in FIG. 1, the operation portion main body 31 also comprises an operation portion 36 for operating the bending portion 235. The operation portion 36 will be described later.

As shown in FIG. 1, the operation portion main body 31 also comprises an operation portion 18b for operating the imaging unit 18 to acquire later-described images D and E.

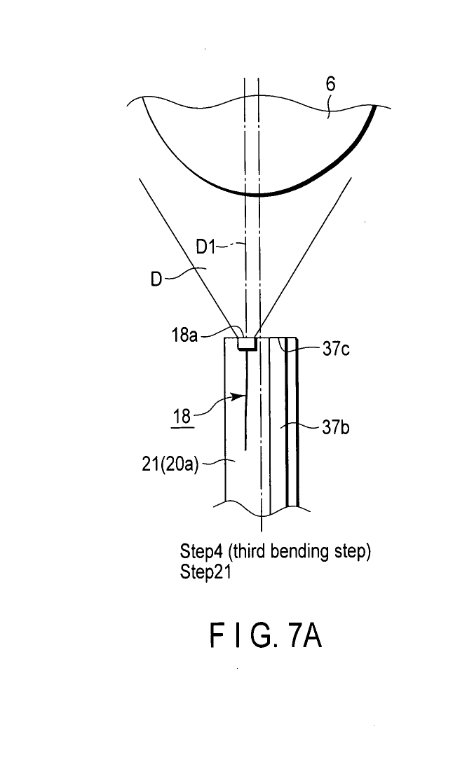

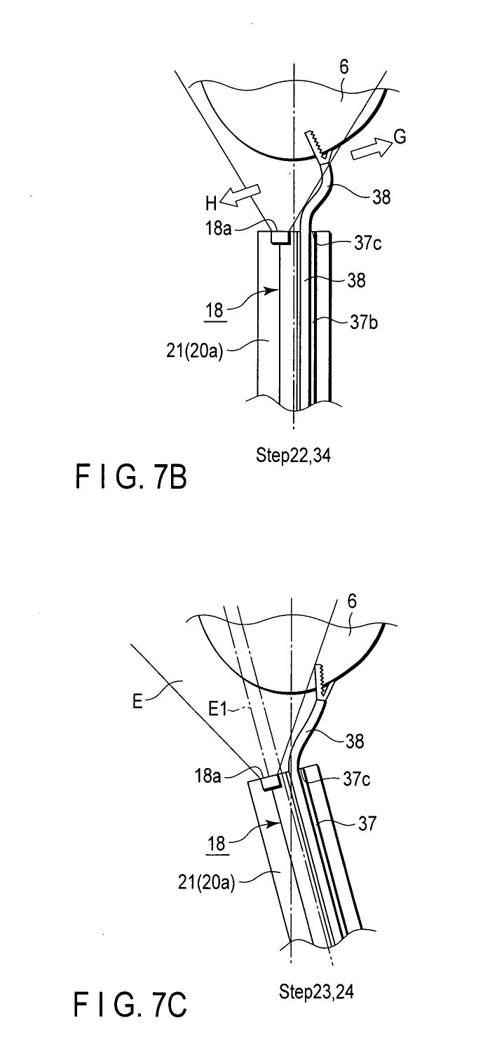

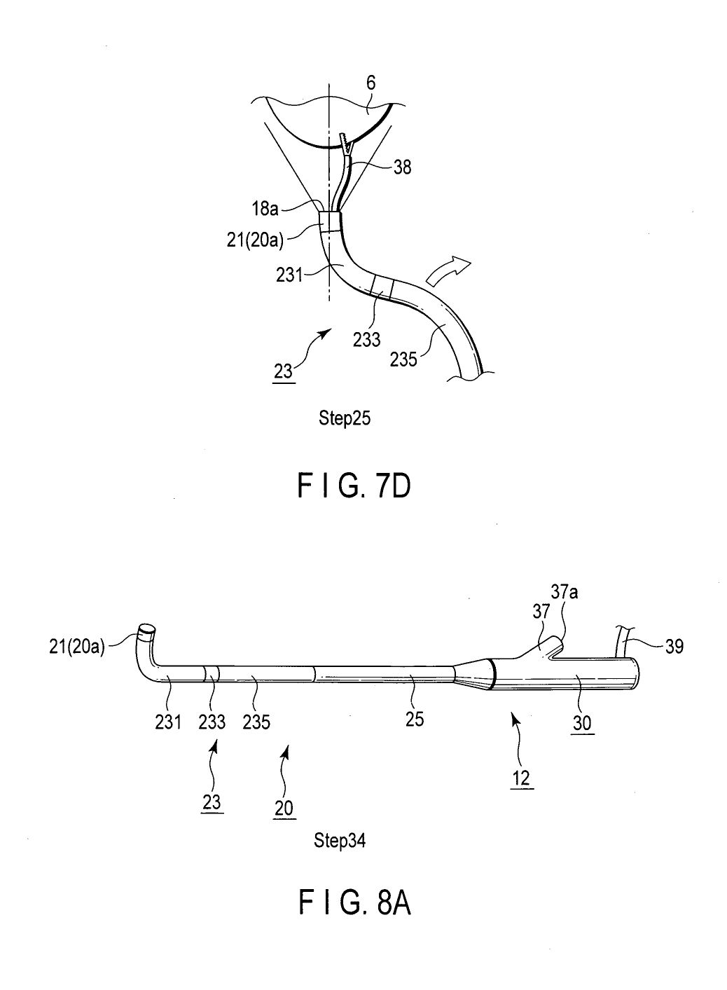

As shown in FIG. 1, the operation portion main body 31 also comprises a surgical instrument insertion portion 37. The surgical instrument insertion portion 37 has a surgical instrument insertion aperture 37a. The surgical instrument insertion aperture 37a is coupled to the proximal end of the surgical instrument insertion channel 37b shown in FIG. 7A. The surgical instrument insertion channel 37b is provided in the insertion portion 20 to extend from the flexible tube 25 to the distal rigid portion 21. That is, the surgical instrument insertion channel 37b is provided in the insertion portion 20. As shown in FIG. 1, the distal opening portion portion 37c which is the distal end of the surgical instrument insertion channel 37b is provided in the distal rigid portion 21.

The surgical instrument insertion aperture 37a is an insertion aperture for inserting an endoscope surgical instrument (hereinafter, a surgical instrument 38) into the surgical instrument insertion channel 37b. The surgical instrument 38 is inserted into the surgical instrument insertion channel 37b from the surgical instrument insertion aperture 37a. After pressed to the distal rigid portion 21, the surgical instrument 38 projects from the distal opening portion portion 37c of the surgical instrument insertion channel 37b provided in the distal rigid portion 21, as shown in FIG. 7B. The surgical instrument 38 is, for example, a pair of forceps.

The universal cord 39 extends from the side surface of the operation portion main body 31. The universal cord 39 has, at its end, a connector 39a attachable to/detachable from the control unit 14.

The endoscope 12 has the imaging unit 18 for imaging the target 6 via the imaging surface 18a. The imaging surface 18a is provided at the distal end 20a of the insertion portion 20 (the distal rigid portion 21).

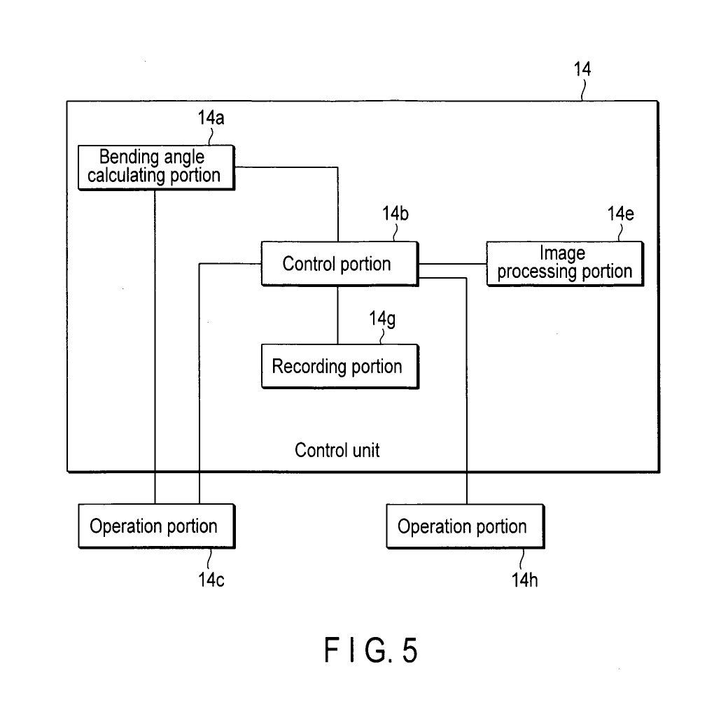

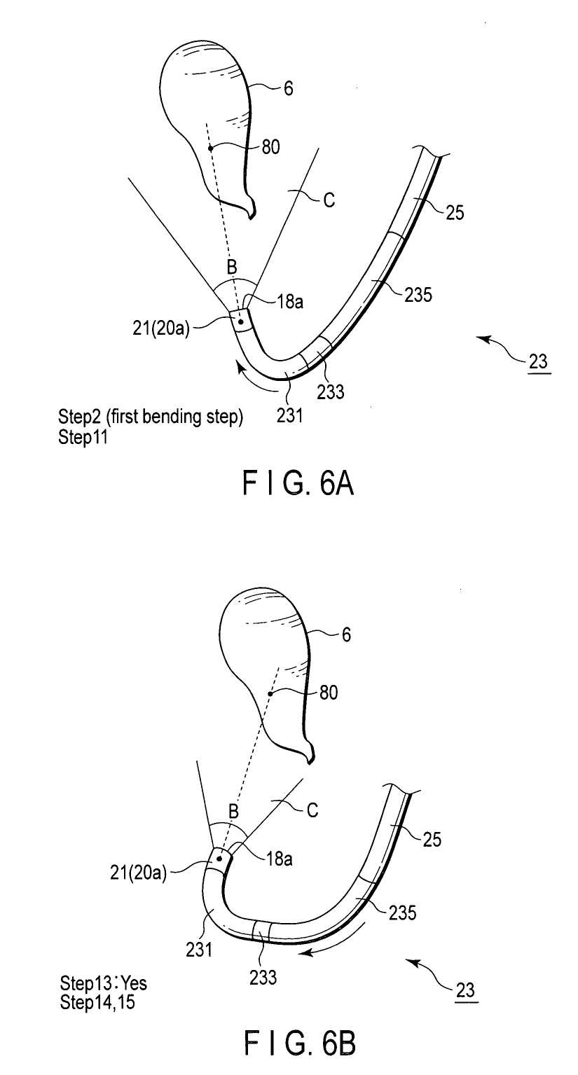

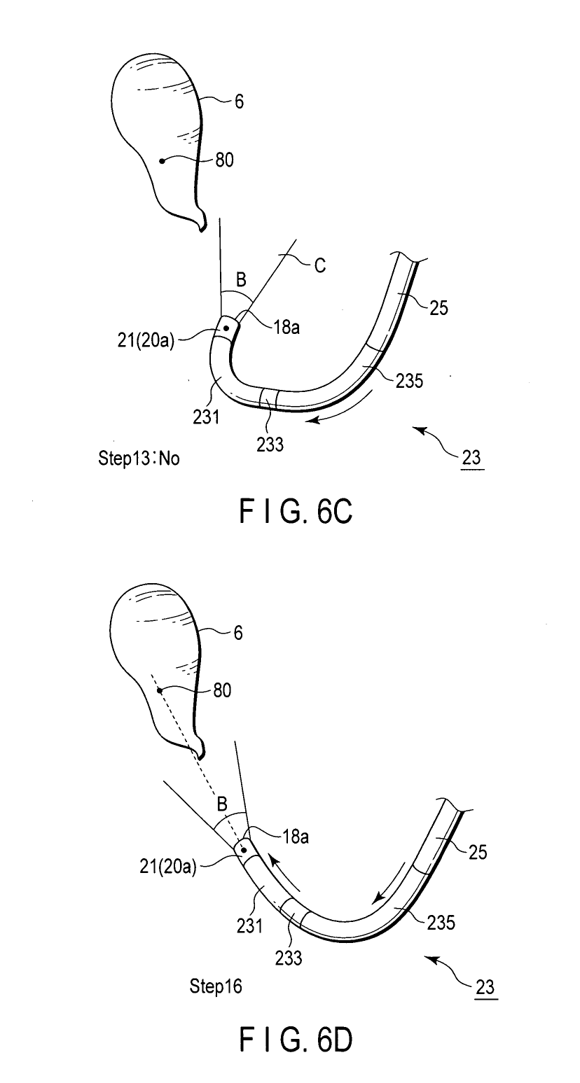

As shown in FIG. 5, the control unit 14 comprises the bending angle calculating portion 14a for calculating the bending angles of the bending portions 231, 233, and 235, and the control portion 14b for controlling the driving portion 333e in accordance with the calculation by the bending angle calculating portion 14a so that the bending portion 235 bent to bring the distal end 20a of the insertion portion 20 closer to a desired point (hereinafter, a target point 80) as shown in FIG. 6A, FIG. 6B, FIG. 6C, and FIG. 6D.

The above-mentioned operation portion 14c operates the bending angle calculating portion 14a so that the bending angle calculating portion 14a calculates the bending angles of the bending portions 231, 233, and 235. The operation portion 14c also operates the control portion 14b so that the control portion 14b sets the target point 80. The operation portion 14c also operates the control portion 14b so that the control portion 14b controls the driving portion 333e as described above to bring the distal end 20a of the insertion portion 20 closer to the target point 80.

The bending angle calculating portion 14a calculates the bending angles of the bending portions 231, 233, and 235, for example, from the bending operation mechanisms of the bending portions 231, 233, and 235 and the driving portion 333e. The bending angle calculating portion 14a has only to calculate the bending angle of the bending portion 233, for example, from at least one of the bending operation mechanism 332d and the driving portion 332e of the bending portion 233. The bending angle calculating portion 14a may calculate the bending angles of the bending portions 231, 233, and 235 from unshown optical sensors or magnetic sensors provided in the bending portions 231, 233, and 235, or from changes in the tension of the operation wire 60. The bending angle calculating portion 14a may calculate the position of the distal end 20a of the insertion portion 20 in accordance with the calculated bending angles of the bending portions 231, 233, and 235.

The distal end 20a of the insertion portion 20 is, for example, the distal rigid portion 21 as described above.

As shown in FIG. 6A, the control portion 14b sets, as the target point 80, a point a desired distance apart from the imaging surface 18a, for example, to the body cavity when the operation portion 14c is operated. In this case, the control portion 14b calculates the position of the target point 80. The target point 80 shown in FIG. 6A represents the point a desired distance apart from the imaging surface 18a, for example, to the body cavity, and is, for example, a part of the target 6 or a part of the body cavity. As shown in FIG. 6A, the target point 80 is located on an imaging screen (an imaging view field angle B, an imaging region C) imaged by the imaging unit 18 (the imaging surface 18a). A desired view field angle is set. The imaging surface 18a is provided in the distal end 20a of the insertion portion 20 (the distal rigid portion 21) as described above. This distance is within the depth of a subject.

The control portion 14b determines in accordance with the calculation (bending angle) by the bending angle calculating portion 14a whether the target point 80 is located on the imaging screen (imaging view field angle B) imaged by the imaging surface 18a when the bending portion 235 is bent.

As shown in FIG. 6B, when the target point 80 is located on the imaging screen (imaging view field angle B), the control portion 14b controls the driving portion 333e in accordance with the calculation by the bending angle calculating portion 14a so that the bending portion 235 bent to bring the distal end 20a of the insertion portion 20 closer to the target point 80, as described above.

In this way, in accordance with the calculation by the bending angle calculating portion 14a, the control portion 14b determines the direction in which the distal end 20a of the insertion portion 20 approaches the target 6 that is an object.

If the operation portion 14c is operated, the control portion 14b controls the driving portion 333e to fix the bending portion 235.

As shown in FIG. 6C, when the target point 80 is located off the imaging screen (imaging view field angle B), the control portion 14b controls the driving portion 333e to fix the electrically driven the bending portion 235. At the same time, as shown in FIG. 6D, at least one of the bending portions 231 and 233 is again bent manually. When the operation portion 14c is operated, the bending angle calculating portion 14a calculates the bending angles of the bending portions 231, 233, and 235. The control portion 14b again determines in accordance with the calculation by the bending angle calculating portion 14a whether the target point 80 is located on the imaging screen when the bending portions 231 and 233 are bent.

Thus, the control portion 14b controls the bending portion 235 to bring the distal end 20a of the insertion portion 20 closer to the target point 80.

The control unit 14 controls the driving portion 333e so that the bending portion 235 bent in the same direction as or in the direction opposite to the bending portion 231 when the operation portion 36 is operated. The bending portion 235 does not necessarily have to exclusively follow the bend of the bending portion 231 and may follow the bend of the bending portion 233. In this case, the bending portion 235 is preset to follow the bend of one of the bending portion 231 and the bending portion 233. The bending portion 235 is preset to bend in one of the same direction and the opposite direction.

As shown in FIG. 5, the control unit 14 comprises an image processing portion 14e for calculating the displacement of the imaging screen, that is, the degree of displacement of the distal end 20a of the insertion portion 20 in accordance with image D (first image) and image E (second image).

As shown in FIG. 7A, image D is obtained by the imaging unit 18 when the operation portion 18b is operated before the surgical instrument 38 projects from the distal opening portion portion 37c of the surgical instrument insertion channel 37b. As shown in FIG. 7C, image E is obtained by the imaging unit 18 while the surgical instrument 38 is projecting a desired amount from the distal opening portion portion 37c of the surgical instrument insertion channel 37b. When the operation portion 18b is operated, the imaging unit 18 keeps imaging, and images are always acquired.

For example, the image processing portion 14e sets a central point D1 of image D from image D, and sets a central point E1 of image E from image E. The image processing portion 14e then calculates, for example, how far central point D1 of image D is displaced in image E from central point E1 of image E. Thus, the image processing portion 14e calculates the degree of displacement of the distal end 20a of the insertion portion 20 in accordance with image D and image E. The image processing portion 14e calculates the degree of displacement of the distal end 20a of the insertion portion 20, and thereby calculates the bending angle and bending direction of the bending portion 235.

In general, as shown in FIG. 7B, if the surgical instrument 38 projects from the distal opening portion portion 37c and moves, reaction force H of the surgical instrument 38 is generated in the distal end 20a of the insertion portion 20 by movement force G of the surgical instrument 38. As a result, the distal end 20a of the insertion portion 20 is displaced by the reaction force H of the surgical instrument 38, as shown in FIG. 7C. That is, the imaging screen in the imaging unit 18 is also displaced.

Therefore, when the surgical instrument 38 projects a desired amount from the distal opening portion portion 37c of the surgical instrument insertion channel 37b as shown in FIG. 7B and then moves to cause the distal end 20a of the insertion portion 20 to be displaced as shown in FIG. 7C, the control portion 14b controls the driving portion 333e in accordance with the degree of displacement calculated by the image processing portion 14e so that the bending portion 235 bent as shown in FIG. 7D and thus image E in which the displacement is eliminated may be image D.

Therefore, even if the surgical instrument 38 projects a desired amount from the distal opening portion portion 37c of the surgical instrument insertion channel 37b, the bending portion 235 bent as shown in FIG. 7D so that the position of the distal end 20a of the insertion portion 20 is corrected to the state before the surgical instrument 38 projects a desired amount from the distal opening portion portion 37c of the surgical instrument insertion channel 37b. This state is the state shown in FIG. 7A.

The degree of displacement of the distal end 20a of the insertion portion 20 may be calculated by the bending angle calculating portion 14a. In this case, the bending angle calculating portion 14a calculates the bending angles of the bending portions 231, 233, and 235 when the operation portion 18b is operated before the surgical instrument 38 projects a desired amount from the distal opening portion portion 37c. The bending angle calculating portion 14a also calculates the bending angles of the bending portions 231, 233, and 235 after the surgical instrument 38 projects a desired amount from the distal opening portion portion 37c.

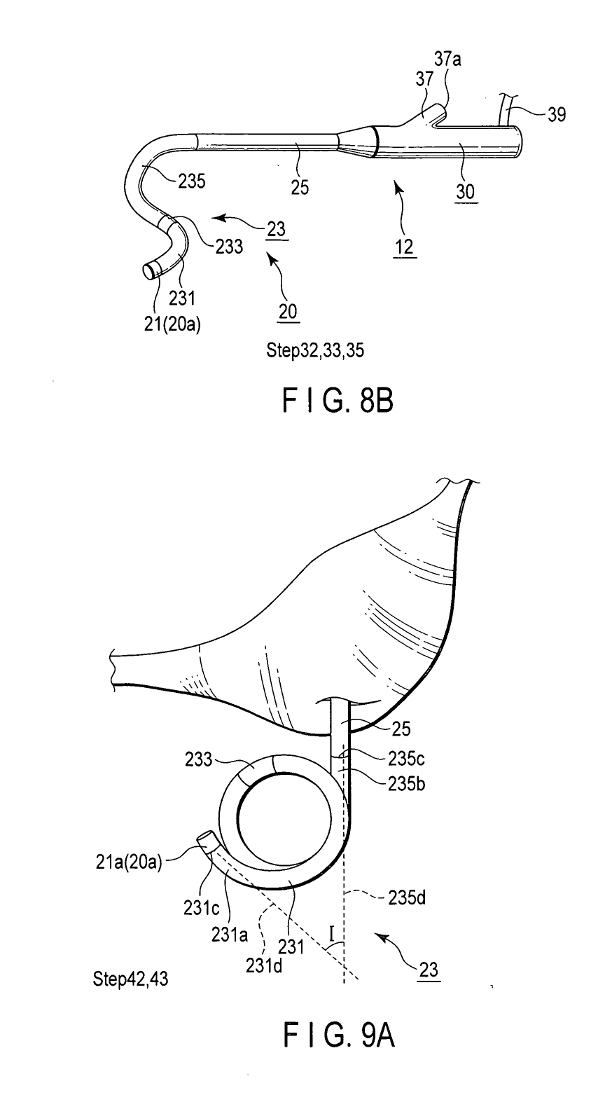

As shown in FIG. 5, the control unit 14 comprises a recording portion 14g which records the bending angle of the bending portion 235 calculated by the bending angle calculating portion 14a when the bending portion 235 is bent as shown in FIG. 8B, and a desired bending angle of the bending portion 235 shown in FIG. 8A.

In the operation portion 14h described above, the surgical instrument insertion/removal mode indicates that the bending portion 235 is bent at the desired bending angle recorded by the recording portion 14g when the surgical instrument 38 is inserted into or removed from the endoscope 12 (the surgical instrument insertion aperture 37a, the surgical instrument insertion channel 37b). At the same time, the bending angle is, for example, 180°, and the bending portion 235 is linear as shown in FIG. 8A. The surgical instrument insertion/removal mode indicates that the bending portion 235 is bent at the bending angle recorded by the recording portion 14g as shown in FIG. 8B after the surgical instrument 38 is inserted into the endoscope 12 (the surgical instrument insertion aperture 37a, the surgical instrument insertion channel 37b).

If the operation portion 14h sets the endoscope 12 to the surgical instrument insertion/removal mode, the bending angle calculating portion 14a calculates the current bending angle of the bending portion 235 which is bent as shown in FIG. 8B, and the recording portion 14g records the bending angle.

After the surgical instrument insertion/removal mode is set, the bending operation portion 333 is operated so that the surgical instrument 38 is inserted into, for example, the surgical instrument insertion channel 37b. As a result, the control portion 14b controls the driving portion 333e so that the bending portion 235 will be at the desired bending angle (linear) recorded by the recording portion 14g. Accordingly, the bending portion 235 becomes linear as shown in FIG. 8A. In this condition, the surgical instrument 38 is inserted into the endoscope 12 (the surgical instrument insertion channel 37b).

When the operation portion 14h cancels the surgical instrument insertion/removal mode, the control portion 14b controls the driving portion 333e so that the bending portion 235 will be bent at the bending angle recorded by the recording portion 14g even if the surgical instrument 38 is inserted into the surgical instrument insertion channel 37b.

The control unit 14 may comprise a detector which detects that the surgical instrument 38 is inserted into or removed from the surgical instrument insertion channel 37b (the surgical instrument insertion aperture 37a). The surgical instrument insertion/removal mode may be set when the detector detects the insertion, and the surgical instrument insertion/removal mode may be canceled when the detector detects the removal.

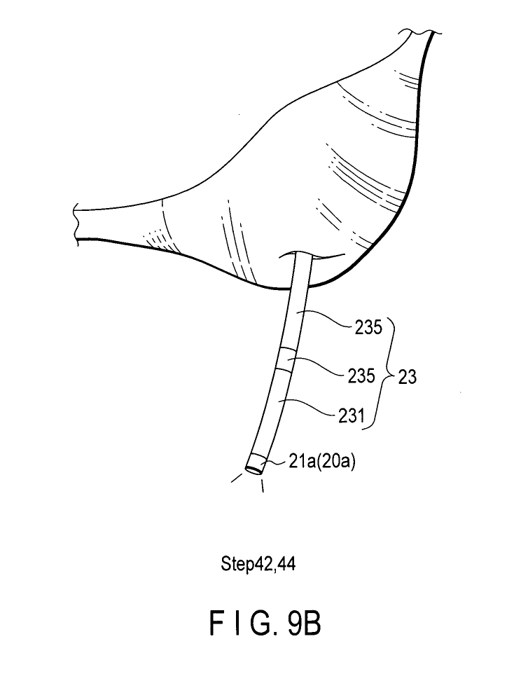

The bending angle calculating portion 14a calculates an overall bending angle I which represents the bending angle of the whole the bending portion. As shown in FIG. 9A, the overall bending angle I is an angle made between a distal straight line 231d in the axial direction of a distal end 231a of the bending portion 231 and a proximal straight line 235d in the axial direction of a proximal end 235b of the bending portion 235.

For example, the plane in the diametrical direction of the node ring 50a is a distal cross section 231c. In this case, the distal straight line 231d is perpendicular to the distal cross section 231c.

For example, the plane in the diametrical direction of the node ring 50 in the bending portion 235 connected to the flexible tube 25 is a proximal cross section 235c. In this case, the proximal straight line 235d is perpendicular to the proximal cross section 235c.

The control unit 14 determines whether the overall bending angle I calculated by the bending angle calculating portion 14a is beyond a desired value. The control portion 14b thereby determines whether the bending portion has made one rotation as shown in FIG. 9A.

When the overall bending angle I is beyond the desired value, that is, when the control portion 14b determines that the bending portion has made one rotation as shown in FIG. 9A, the control portion 14b controls the driving portion 333e so that the bending portion 235 will not bend any more. As a result, the bend of the bending portion 235 is limited.

The monitor 16 displays the image obtained by the imaging surface 18a.

Now, an operating method according to the present embodiment is described.

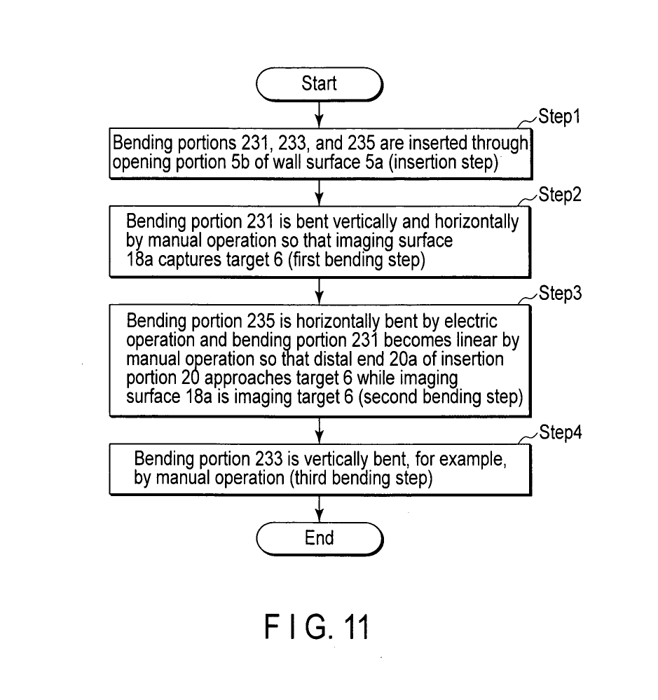

First, the operating method of the whole endoscope 12 is described with reference to FIG. 10A, FIG. 10B, FIG. 10C, FIG. 10D, and FIG. 11.

A wall surface 5a of a natural orifice organ 5 (affected part) is opened by an unshown surgical instrument.

As shown in FIG. 10A, the insertion portion 20 including the bending portions 231, 233, and 235 is inserted into a body cavity (abdominal cavity). The bending portions 231, 233, and 235 are then inserted through an opening portion 5b of the wall surface 5a (Step 1, insertion step).

As shown in FIG. 10B, the horizontal bending operation knob 331a and the vertical bending operation knob 331b are then operated so that the imaging surface 18a captures (images) the target 6 (for example, the gallbladder). As a result, in the bending portion 231, the horizontal operation wire 60 is pulled via the horizontal bending operation mechanism, and the vertical operation wire 60 is pulled via the vertical bending operation mechanism. Thus, the bending portion 231 is bent vertically and horizontally by manual operation (Step 2, first bending step).

As shown in FIG. 10B, the bending portion 231 is manually operated and bent vertically and horizontally. Therefore, the bending portion 231 bent more finely than the bending portions 233 and 235, and the distal end 20a of the insertion portion 20 finely approaches the target 6, and then the imaging surface 18a easily captures the target 6.

As shown in FIG. 10C, the bending operation portion 333 then operates the driving portion 333e to bring the distal end 20a of the insertion portion 20 closer to the target 6 while the imaging surface 18a is imaging the target 6. Accordingly, in the bending portion 235, the operation wire 60 is pulled via the driving force of the driving portion 333e and the horizontal bending operation mechanism. The bending portion 235 is then horizontally bent by the driving force of the driving portion 333e and the horizontal bending operation mechanism. The bending portion 235 is thus horizontally bent by electric operation. The bending angle of the bending portion 235 is the same as the bending angle of the bending portion 231 and more than the bending angle of the bending portion 233. The bending portion 235 is longer than the bending portions 231 and 233. Therefore, the bending portion 235 bent more widely than the bending portions 231 and 233. The distal end 20a of the insertion portion 20 roughly approaches the target 6.

When the bending portion 235 is bent, the horizontal bending operation knob 331a and the vertical bending operation knob 331b are operated so that the bending portion 231 becomes, for example, linear as shown in FIG. 10C in order for the imaging surface 18a to keep capturing the target 6 and in order to prevent the target 6 from being located out of the view field of the imaging surface 18 due to the bend of the bending portion 235. As a result, the horizontal operation wire 60 is pulled via the horizontal bending operation mechanism, and the vertical operation wire 60 is pulled via the vertical bending operation mechanism. Thus, the bending portion 231 becomes, for example, linear by manual operation (Step 3, second bending step).

The electrically driven the bending portion 235 bent in this way, so that the burden on the surgeon is reduced. In addition, the distal end 20a of the insertion portion 20 roughly approaches the target 6.

In the manual operation, the bending portion 231 having a larger bending angle than the bending portion 233 becomes linear. Thus, even if the bending portion 235 is bent, the imaging surface 18a can keep capturing the target 6 without losing sight of the target 6. Once the bending portion 231 is restored to the linear state, the bending portion 231 is again able to bend, and the position of the distal end 20a of the insertion portion 20 is finely adjusted.

As shown in FIG. 10D, for example, the vertical bending operation knob 332b is then operated, and the operation wire 60 is pulled via the vertical bending operation mechanism. Thus, the bending portion 233 is vertically bent, for example, by manual operation (Step 4, third bending step).

The bending portion 233 shorter than the bending portions 231 and 235 is vertically bent by manual operation in the end. As a result, the position of the distal end 20a of the insertion portion 20 is finely adjusted, and the distal end 20a of the insertion portion 20 finely approaches the target 6. Moreover, the bending portion 233 vertically bent so that the height position of the imaging surface 18a is adjusted.

The driving portion 332e may be attached to the operation portion main body 31 to drive the bending portion 233. Thus, the bending portion 233 is changed to electric operation depending on the target 6 and the surgeon. A desired electric bending direction of the bending portion 23 is selected by the bending portions 233 and 235.

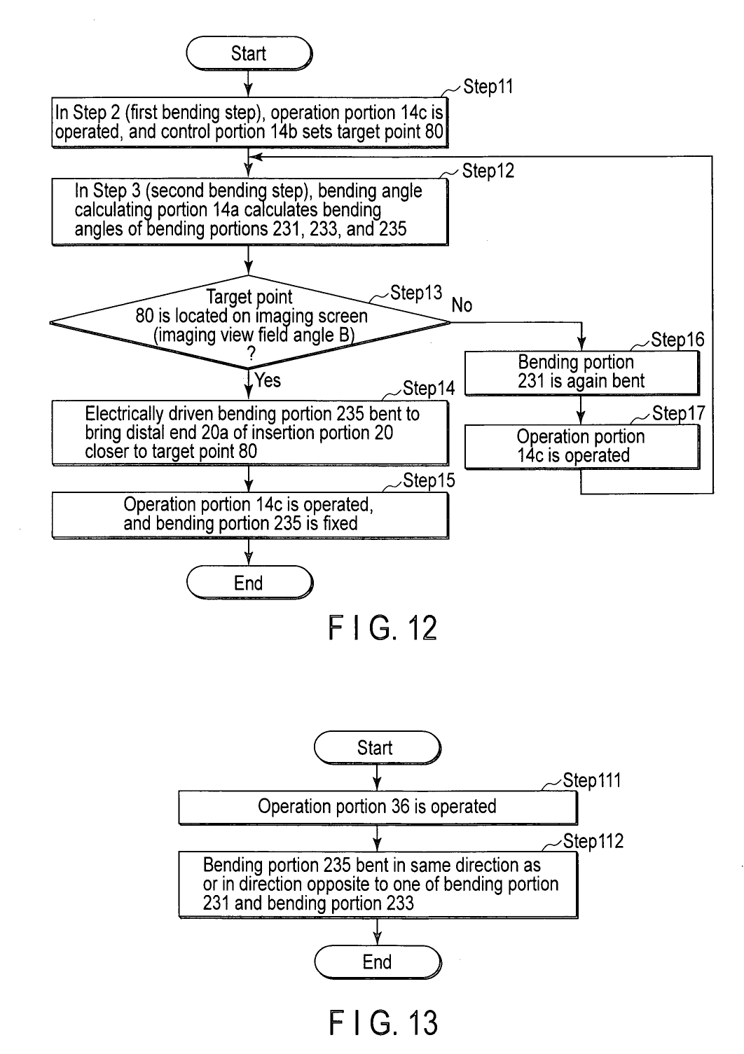

Now, how the bending portion 235 is bent when the target 6 is imaged is described with reference to FIG. 6A, FIG. 6B, FIG. 6C, FIG. 6D, and FIG. 12.

In Step 2 (first bending step) shown in FIG. 6A and FIG. 10B, when the bending portion 231 is bent and the imaging surface 18a captures the target 6, the operation portion 14c is operated, and the control portion 14b sets the target point 80 (Step 11). For example, in Step 2 (first bending step), the target point 80 is the target 6 imaged by the imaging surface 18a.

In Step 3 (second bending step) shown in FIG. 10C, when, for example, the bending portion 235 is bent, the operation portion 14c is operated. Thus, the bending angle calculating portion 14a calculates the bending angles of the bending portions 231, 233, and 235 (Step 12).

As shown in FIG. 6B and FIG. 6C, the control portion 14b determines in accordance with the bending angles calculated by the bending angle calculating portion 14a whether the target point 80 is located on the imaging screen (imaging view field angle B) (Step 13).

As shown in FIG. 6B, when the target point 80 is located on the imaging screen (imaging view field angle B) (Step 13: Yes), the control portion 14b controls the driving portion 333e in accordance with the calculation by the bending angle calculating portion 14a as shown in FIG. 10C. Accordingly, the electrically driven the bending portion 235 further bent to bring the distal end 20a of the insertion portion 20 closer to the target point 80 (Step 14).

When the operation portion 14c is operated, the control portion 14b controls the driving portion 333e to fix the bending portion 235 (Step 15).

As shown in FIG. 6C, when the target point 80 is located off the imaging screen (imaging view field angle B) (Step 13: No), the control portion 14b controls the driving portion 333e to fix the electrically driven the bending portion 235. At the same time, as shown in FIG. 6D, for example, the horizontal bending operation knob 331a and 331b are again operated, and the bending portion 231 is again manually bent (Step 16).

When the operation portion 14c is operated (Step 17), the flow returns to Step 12, and the bending angle calculating portion 14a calculates the bending angles of the bending portions 231, 233, and 235.

Thus, the control portion 14b controls the bending portion 235 to bring the distal end 20a of the insertion portion 20 closer to the target point 80.

In accordance with the calculation by the bending angle calculating portion 14a, the control portion 14b determines the direction in which the distal end 20a of the insertion portion 20 approaches the target 6 that is an object (target point 80). The control portion 14b then controls the driving portion 333e, and controls the bending direction. Therefore, in Step 3 (second bending step), the user does not need to bend the bending portion 235 by manual operation to bring the distal end 20a of the insertion portion 20 closer to the target 6. The user does not need to consider the bending direction of the bending portion 235 either. Thus, the burden on the surgeon is reduced.

Now, how the bending portion 235 is bent is described with reference to FIG. 13.

For example, in Step 3 (second bending step), the operation portion 36 is operated (Step 111).

Accordingly, the control unit 14 controls the driving portion 333e so that the bending portion 235 bent in the same direction as or in the direction opposite to one of the bending portion 231 and the bending portion 233. As a result, the bending portion 235 bent in the same direction as or in the direction opposite to one of the bending portion 231 and the bending portion 233 (Step 112).

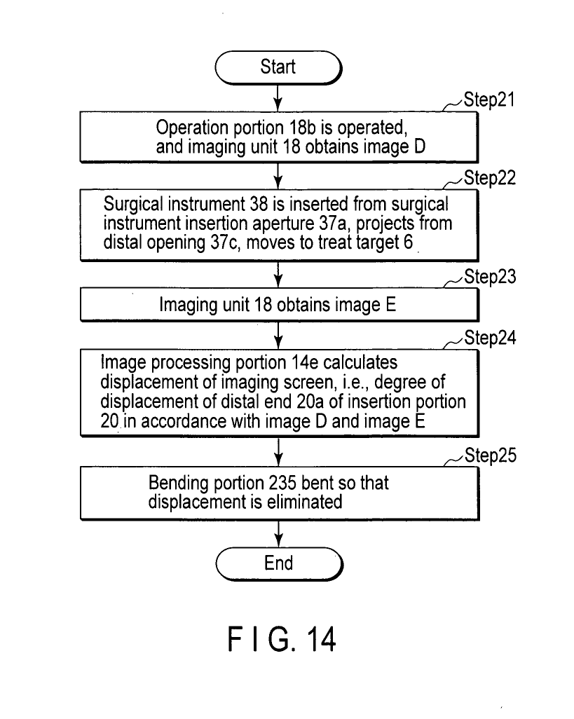

Now, how the bending portion 235 is bent when the surgical instrument 38 moves is described with reference to FIG. 7A, FIG. 7B, FIG. 7C, FIG. 7D, and FIG. 14.

After Step 4 (third bending step) shown in

FIG. 10D, the operation portion 18b is operated, and the imaging unit 18 obtains image D as shown in FIG. 7A (Step 21).

As shown in FIG. 7B, the surgical instrument 38 is then inserted into the surgical instrument insertion channel 37b from the surgical instrument insertion aperture 37a, projects from the distal opening portion portion 37c, and moves to treat the target 6 (Step 22). When the surgical instrument 38 moves, the distal end 20a of the insertion portion 20 is displaced by the reaction force H of the surgical instrument 38, as shown in FIG. 7B. Accordingly, the imaging screen in the imaging unit 18 is also displaced.

At the same time, as shown in FIG. 7C, the imaging unit 18 obtains image E (Step 23).

Therefore, the image processing portion 14e calculates the displacement of the imaging screen, that is, the degree of displacement of the distal end 20a of the insertion portion 20 in accordance with image D and image E (Step 24).

The control portion 14b controls the driving portion 333e in accordance with the degree of displacement calculated by the image processing portion 14e. Further, as shown in FIG. 7D, the bending portion 235 bent so that image E in which the displacement is eliminated may be image D (Step 25).

As a result, the displacement of the distal end of the endoscope 12 caused by the movement of the surgical instrument 38 (displacement of the imaging screen) is eliminated, and the operability of the surgical instrument 38 is improved.

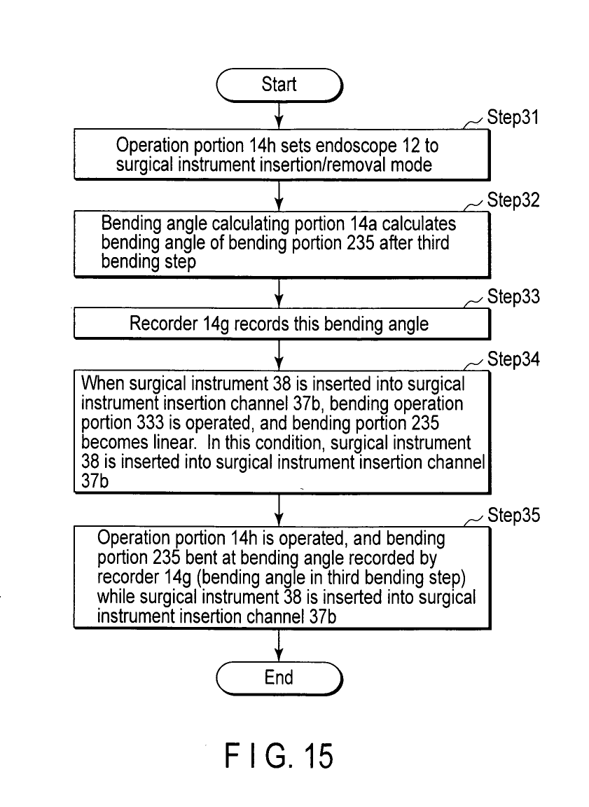

Now, how the bending portion 235 is bent when the surgical instrument 38 is inserted into the surgical instrument insertion channel 37b is described with reference to FIG. 8A, FIG. 8B, and FIG. 15.

After Step 4 (third bending step) shown in FIG. 10D, the operation portion 14h is operated, and the operation portion 14h sets the endoscope 12 to the surgical instrument insertion/removal mode (Step 31).

Thus, as shown in FIG. 8B and FIG. 10D, the bending angle calculating portion 14a calculates the current bending angle of the bending portion 235, that is, the bending angle of the bending portion 235 in Step 4 (after the third bending step) shown in FIG. 10D (Step 32).

The recording portion 14g records this bending angle (Step 33).

When the surgical instrument 38 is inserted into the surgical instrument insertion channel 37b in Step 22, the bending operation portion 333 is operated, and the control portion 14b controls the driving portion 333e so that the bending portion 235 will be linear (bend at a desired bending angle) as shown in FIG. 8A. In this condition, the surgical instrument 38 is inserted into the surgical instrument insertion channel 37b (Step 34).

When operation portion 14h is operated and cancels the surgical instrument insertion/removal mode, the control portion 14b controls the driving portion 333e so that the bending portion 235 bent at the bending angle recorded by the recording portion 14g as shown in FIG. 8B and FIG. 10D (the bending angle in Step 4 [third bending step]) while the surgical instrument 38 is inserted into the surgical instrument insertion channel 37b (Step 35).

Thus, after Step 4 (third bending step), the bending portion 235 becomes linear as shown in FIG. 8A when the surgical instrument 38 is inserted into the surgical instrument insertion channel 37b. While the surgical instrument 38 is inserted into the surgical instrument insertion channel 37b, the bending portion 235 bent in the state after Step 4 (third bending step) as shown in FIG. 8B and FIG. 10D, and the surgical instrument 38 treats the target 6.

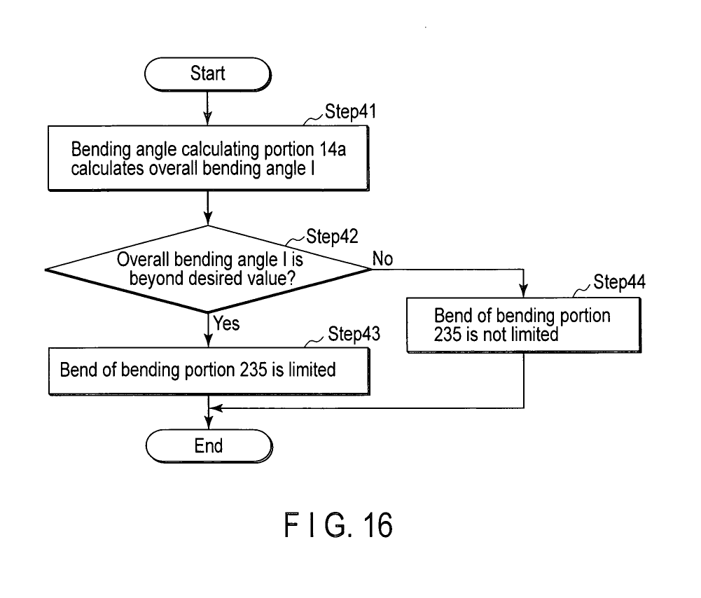

Now, how the control portion 14b controls when the bending portion 23 is bent is described with reference to FIG. 9A, FIG. 9B, and FIG. 16.

After Step 4 (third bending step) shown in FIG. 10D, the bending angle calculating portion 14a calculates the overall bending angle I (Step 41).

The control unit 14 determines whether the overall bending angle I calculated by the bending angle calculating portion 14a is beyond a desired value, and determines whether the bending portion 23 is linear as shown in FIG. 9B or has made one rotation as shown in FIG. 9A (Step 42).

When determining that the overall bending angle I is beyond the desired value, that is, when determining that the bending portion has made one rotation (Step 41: Yes), the control portion 14b controls the driving portion 333e to limit the bend of the bending portion 235 (Step 43). As a result, the bending portion 235 will not bend any more.

When determining that the overall bending angle I is not beyond the desired value, that is, when the bending portion has not made one rotation (Step 41: No), the control portion 14b does not limit the bend of the bending portion 235 (Step 44).

The control portion 14b thereby prevents the bending portion 23 from being erroneously recognized as linear even if bent.

In this way, according to the present embodiment, at least three the bending portions 231, 233, and 235 which bend are provided, so that the distal end 20a of the insertion portion 20 can be easily brought closer to the target 6 (for example, the gallbladder), for example, in the NOTES procedure.

Furthermore, according to the present embodiment, the bending portion 235 is longer than the bending portion 233. Thus, the distal end 20a of the insertion portion 20 can be roughly brought closer to the target 6 by the bending portion 235, and the distal end 20a of the insertion portion 20 can be finely brought closer to the target 6 by the bending portion 233.

Still further, according to the present embodiment, the distal end 20a of the insertion portion 20 can be finely brought closer to the target 6 by vertically and horizontally bending the bending portion 231, the height position of the imaging surface 18a can be adjusted by vertically bending the bending portion 233, and the distal end 20a of the insertion portion 20 can be roughly brought closer to the target 6 by horizontally bending the bending portion 235.

Still further, according to the present embodiment, the distal end 20a of the insertion portion 20 can be finely brought closer to the target 6 by manually bending the bending portion 231. According to the present embodiment, if the bending portion 235 is bent by electric operation, the burden on the surgeon can be reduced. According to the present embodiment, if the bending portion 235 is bent by electric operation, the surgeon can concentrate on the operation of the bending portion 231 without being bothered by the operation of the bending portion 235, and can simultaneously operate the bending portion 231 and the bending portion 235 in Step 3 (second bending step). According to the present embodiment, if the bending portion 235 is bent by electric operation, the bending operation portion 333 which is a switch has only to be provided in the operation portion main body 31, and the operation knob provided to operate the bending portion 235 can be dispensed with. According to the present embodiment, it is thus possible to reduce the operation portion main body 31 in size.

Still further, according to the present embodiment, the length of the bending portion 231 is 85 mm, so that the distal end 20a of the insertion portion 20 can be finely brought closer to the target 6 vertically and horizontally by the bending portion 231. According to the present embodiment, the length of the bending portion 233 is 45 mm, so that the distal end 20a of the insertion portion 20 can be finely brought closer to the target 6 vertically by the bending portion 233. According to the present embodiment, the length of the bending portion 235 is 90 mm, so that the distal end 20a of the insertion portion 20 can be roughly brought closer to the target 6 horizontally by the bending portion 235.

Still further, according to the present embodiment, the bending angle of the bending portion 231 is 150° or more and 210° or less, so that the target 6 can be easily captured by the imaging surface 18a when the bending portions 231, 233, and 235 are inserted through the opening portion. According to the present embodiment, the bending angle of the bending portion 233 is 60° or more and 80° or less, so that the distal end 20a of the insertion portion 20 can be easily brought closer to the target 6, and the height position of the imaging surface 18a can be adjusted. According to the present embodiment, the bending angle of the bending portion 235 is 150° or more and 210° or less, so that the distal end 20a of the insertion portion 20 can be easily brought closer to the target 6 while the imaging surface 18a keeps the target 6 captured.

Still further, according to the present embodiment, if the driving portion 332e is attached to the operation portion main body 31, the bending portion 233 can be easily bent electrically. Thus, according to the present embodiment, the bending portion 233 is changed to electric operation or manual operation depending on the target 6 and the surgeon. Moreover, according to the present embodiment, a desired electric bending direction of the bending portion 23 can be selected by the bending portions 233 and 235.

Still further, according to the present embodiment, the distal end 20a of the insertion portion 20 can be brought closer to the target 6 (target point 80) by the bending angle calculating portion 14a and the control portion 14b in Step 3 (second bending step), and the bending portion 235 does not need to be bent by manual operation. Moreover, according to the present embodiment, the burden on the surgeon can be reduced.

Still further, according to the present embodiment, for example, in Step 3 (second bending step), the bending portion 235 can be bent in the same direction as or in the direction opposite to one of the bending portion 231 and the bending portion 233, and there is no need to consider the bending direction of the bending portion 235, so that the burden on the surgeon can be reduced.

Still further, according to the present embodiment, when the surgical instrument 38 moves in a state projecting a desired amount from the distal opening portion portion 37c, the displacement of the distal end of the endoscope 12 caused by the movement of the surgical instrument 38 (displacement of the imaging screen) can be eliminated by the image processing portion 14e and the control portion 14b, and the operability of the surgical instrument 38 can be improved.

Still further, according to the present embodiment, after Step 3 (second bending step), the bending portion 235 can be linear by the bending angle calculating portion 14a, the recording portion 14g, and the control portion 14b, and the surgical instrument 38 can be easily inserted into the surgical instrument insertion channel 37b. Moreover, according to the present embodiment, the bending portion 235 can be bent (returned) to the state after Step 4 (third bending step) while the surgical instrument 38 is inserted into the surgical instrument insertion channel 37b. Thus, the target 6 can be quickly treated by the surgical instrument 38.

Still further, according to the present embodiment, the overall bending angle I can be calculated by the bending angle calculating portion 14a. Thus, according to the present embodiment, the control portion 14b can determine the bending state of the bending portion, that is, the control portion 14b can prevent the bending portion 23 from being erroneously recognized as linear even if bent. Therefore, according to the present embodiment, it is possible to prevent the bending portion 23 which is bent 360° from being removed from an opening portion, and reduce the patient's pain during the removal of the insertion portion 20.

Still further, according to the present embodiment, the bending operation portion 333 is provided between the horizontal bending operation knob 331a, the vertical bending operation knob 331b, and the vertical bending operation knob 332b, and the exterior of the endoscope 12, so that the bending portions 231, 233, and 235 can be bent by one hand.

Still further, according to the present embodiment, the bending portion 235 is electrically bent, and there is thus no need for, for example, an over tube, thereby allowing a simpler operation system and allowing the position of the distal end 20a of the insertion portion 20 to be easily known.

The present invention is not completely limited to the embodiment described above, and modifications of components can be made at the stage of carrying out the invention without departing from the spirit thereof. Further, various inventions can be made by properly combining the components disclosed in the embodiment described above.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an insertion portion having a longitudinal axis;
a bending portion which is provided at the distal end of the insertion portion and which is configured to bend;
a holding portion which is provided closer to the distal end of the insertion portion than the bending portion and which holds the distal end of a surgical instrument so that the distal end of the surgical instrument is configured to project from the distal end of the insertion portion;
a driving portion which drives the bending portion;
a degree of displacement calculating portion which calculates a degree of displacement of the distal end of the insertion portion when the distal end of the insertion portion is displaced by reaction force generated by the movement of the distal end of the surgical instrument projecting from the distal end of the insertion portion; and
a control portion which controls a driving amount of the driving portion to eliminate the displacement in accordance with the degree of displacement calculated by the degree of displacement calculating portion.

2. The endoscope according to claim 1, further comprising: an imaging unit which is provided at the distal end of the insertion portion and which images a target,
wherein the degree of displacement calculating portion comprises an image processing portion which calculates the degree of displacement of the distal end of the insertion portion in accordance with a first image of the target obtained by the imaging unit and in accordance with a second image obtained by the imaging unit when the distal end of the insertion portion is displaced by reaction force generated by the movement of the distal end of the surgical instrument projecting from the distal end of the insertion portion.

3. The endoscope according to claim 2, wherein the driving portion drives the proximal end side of the bending portion.

4. The endoscope according to claim 3, characterized in that the insertion portion comprises a surgical instrument insertion channel through which to insert the surgical instrument, and
the holding portion is a distal opening portion which is in communication with the surgical instrument insertion channel and which is provided at the distal end of the insertion portion.

5. The endoscope according to claim 4, wherein the bending portion comprises
a first bending portion which is provided at the distal end of the insertion portion and which bent vertically and horizontally,
a second bending portion which is coupled to the proximal end of the first bending portion and which bent vertically, and
a third bending portion which is coupled to the proximal end of the second bending portion and which is longer than the second bending portion in a longitudinal axis direction and which bent horizontally, and
the driving portion drives the third bending portion.

6. The endoscope according to claim 5, wherein
the first bending portion is bent by manual operation,
the second bending portion is bent by manual operation or electric operation, and
the third bending portion is bent by electric operation.

7. The endoscope according to claim 6, wherein the length of the first bending portion is 85 mm, the length of the second bending portion is 45 mm, and the length of the third bending portion is 90 mm.

8. The endoscope according to claim 7, wherein the first bending portion, the second bending portion, and the third bending portion each comprises a proximal straight line in the axial direction of its proximal end, a distal straight line in the axial direction of its distal end, and a maximum bending angle which represents an angle made between the proximal straight line and the distal straight line when each the bending portion is bent,
the maximum bending angle of the first bending portion is 150° or more and 210° or less when the first bending portion is bent,
the maximum bending angle (A) of the second bending portion is 60° or more and 80° or less when the second bending portion is bent, and
the maximum bending angle (A) of the third bending portion is 150° or more and 210° or less when the third bending portion is bent.

9. The endoscope according to claim 8, further comprising: an operation portion main body which manually operates the second bending portion; and
an attachment/detachment driving portion which is attachable to/detachable from the operation portion main body and which has driving force to electrically bend the second bending portion when the second bending portion is bent by electric operation.

10. The endoscope according to claim 5, further comprising: a bending angle calculating portion which calculates the bending angle of the first bending portion, the bending angle of the second bending portion, and the bending angle of the third bending portion,
the control portion sets, as a target point, a point which is located on an imaging screen imaged by the imaging unit and which is a desired distance apart from the imaging surface, and the control portion controls the driving portion in accordance with a calculation by the bending angle calculating portion so that the third bending portion bent to bring the distal end of the insertion portion closer to the target point.

11. The endoscope according to claim 10, wherein the control portion determines in accordance with the calculation whether the target point is located on the imaging screen.

12. The endoscope according to claim 10, wherein when the third bending portion bent, the control portion controls the driving portion so that the third bending portion bent in the same direction as or in the direction opposite to one of the first bending portion and the second bending portion.

13. The endoscope according to claim 10, further comprising: a recording portion which records the bending angle of the third bending portion calculated by the bending angle calculating portion when the third bending portion is bent, and a desired bending angle of the third bending portion,
wherein the control portion controls the driving portion so that the third bending portion is bent at the desired bending angle when the surgical instrument is inserted into the surgical instrument insertion channel, and the control portion controls the driving portion so that the third bending portion is bent at the bending angle recorded by the recording portion when the surgical instrument is inserted into the surgical instrument insertion channel.

14. The endoscope according to claim 10, wherein the bending angle calculating portion calculates an overall bending angle (I) which represents the bending angle of the whole the bending portion, and
the control portion determines whether the overall bending angle (I) calculated by the bending angle calculating portion is beyond a desired value, and when the overall bending angle (I) is beyond the desired value, the control portion controls the driving portion so that the third bending portion is not bent.
